(19) United States
(12) Patent Application Publication
(10) Pub. No.: US 2005/0182145 A1
(43) Pub. Date: Aug. 18, 2005

(54) Methods of reducing and loading a metal-based catalyst into a reactor

(75) Inventors: Sergio R. Mohedas, Ponca City, OK (US); Rafael L. Espinoza, Ponca City, OK (US); Ajoy Raje, Stillwater, OK (US); Kevin L. Coy, Ponca City, OK (US)

Correspondence Address: DAVID W. WESTPHAL;CONOCOPHILLIPS COMPANY - I.P. Legal, P.O. BOX 1267, PONONCA CITY, OK, 74602-1267, US

(73) Assignee: ConocoPhillips Company, Houston, TX (US)

(21) Appl. No.: 10/780,049

(22) Filed: Feb. 17, 2004

(51) Int. Cl. C07C 27/06

(52) U.S. Cl. 518/716; 518/726

(57) ABSTRACT

The present methods feature an overall decrease in transportation costs and catalyst preparation/protection measures. A catalyst comprising a catalytic metal in an oxide form is safely transported in an oxidizing environment to a synthesis site, without any special precautions being taken before and during transport. The catalyst is then reduced with a reducing gas at the synthesis plant. The reduced catalyst is mixed with a stripped hydrocarbon liquid to form a catalyst slurry, wherein the stripped hydrocarbon liquid is substantially free of dissolved oxygen after being contacted with a stripping gas. The mixing can take place in a pre-operational hydrocarbon synthesis reactor, or at least a portion of the slurry can be transferred to at least one synthesis reactor either during operation or at the reactor start-up. A lessening of costs is realized as a coating step to minimize oxidative degradation of the catalyst is not required.

### FIELD OF THE INVENTION

The present invention relates to hydrocarbon synthesis reactors and hydrocarbon synthesis catalysts. In particular, the present invention relates to fluidized bed hydrocarbon synthesis reactors and methods of reducing a metal-based catalyst for use in said reactors and methods of loading said catalyst to said reactors.

### BACKGROUND OF THE INVENTION

Large quantities of natural gas are available in many areas of the world, and natural gas is predicted to outlast oil reserves by a significant margin. However, natural gas reserves have been found in remote areas where economical utilization is limited due to the high cost of transporting the gas to distant markets and/or the lack of local markets for the gas. However, dwindling world petroleum reserves have been partially responsible for efforts to develop economical solutions for these stranded gas reserves. One approach is to locally convert the natural gas to liqud products that can be transported more cost effectively. Processes for converting light hydrocarbon gases, such as natural gas, to heavier hydrocarbon liquids are generally known in the art. One such process, commonly known as the gas to liquids (GTL) process, comprises a two-step process. The first step involves the conversion of natural gas to a mixture of carbon monoxide (CO) and hydrogen (H₂), commonly known as synthesis gas or syngas. The first step to produce synthesis gas can employ for example catalytic and non-catalytic partial oxidation, steam reforming, dry reforming, autothermal reforming, and advanced gas heated reforming. For the second step, synthesis gas can serve as a feedstock for a chemical transformation known as the Fischer-Tropsch synthesis, in which carbon monoxide (CO) and hydrogen (H₂) undergo a reaction, under appropriate conditions of temperature and pressure and in the presence of a suitable catalyst, to form a product stream comprising a mixture of hydrocarbons.

The Fischer-Tropsch product stream typically comprises, at reaction conditions, gaseous and liquid hydrocarbons having a range of molecular weights, with the precise nature of the product stream being dependent upon reaction conditions and catalyst composition. Although both branched and linear hydrocarbons may be present in the product stream, a majority of linear hydrocarbons usually exists in the low-temperature Fischer-Tropsch product stream, where the Fischer-Tropsch reaction typically takes place between 200 and 280° C.

Each hydrocarbon in the Fischer-Tropsch product stream can be characterized by the number of carbon atoms present, and hydrocarbons can be grouped into fractions based on a common carbon number. The composition of the Fischer-Tropsch product stream is greatly affected by the selection of the catalyst as well as by the reaction operating conditions. Since most of the hydrocarbons present in the Fischer-Tropsch product stream are linear hydrocarbons, and since the catalytic synthesis of these hydrocarbons can be conceived in terms of a growing hydrocarbon chain, it is possible to apply polymerization kinetics, which similarly feature a mechanism that invokes a stepwise chain growth mechanism. The well-established statistical treatment that may be used in such cases furnishes a plot of chain growth probability as a function of chain length. The plot of such a correlation is known as an Anderson-Shultz-Flory plot and the slope as the Anderson-Shultz-Flory value, α, or more simply, the alpha value. The Anderson-Schultz-Flory plot is a statistical relationship characterizing the distribution of hydrocarbons obtained from a Fischer-Tropsch synthesis, and the alpha value is defined as the probability of chain growth step to the next higher carbon number divided by the sum of the growth step probability plus the chain termination probability. This parameter can be used to estimate the distribution of carbon number products and thereby the effectiveness of the process to furnish a product stream having a particular carbon number distribution. The alpha value depends on the nature of the catalyst and the fundamental operating conditions to which the catalyst is exposed, such as temperature, pressure, the concentration and the ratio of constituent gases within the reactant gas. Reaction conditions can be adjusted to provide a Fischer-Tropsch product stream comprising a plurality of hydrocarbons, which are gaseous, liquid or waxy hydrocarbons at atmospheric temperature and pressure. It has been a long-standing goal within the art to obtain liquid hydrocarbons from C₁-C₅ gaseous hydrocarbons, such as natural gas, in particular naphtha comprising about C₄-C₁₂ fractions and middle distillates comprising about C₁₀-C₂₂ fractions, via the Fischer-Tropsch process. These highly prized fractions are the source for important transportation fuels such as diesel, gasoline and aviation fuels.

One approach to optimizing the yield of middle distillates from the Fischer-Tropsch process has been to adjust reactor conditions to maximize the direct production of liquid hydrocarbons. Alternatively, some in the art have sought to obtain high yields of middle distillates by processing a Fischer-Tropsch product that comprises a plurality of waxy hydrocarbons under suitable conditions. According to one method, obtaining such a Fischer-Tropsch wax may comprise initially separating lighter gaseous and liquid hydrocarbons from the desired wax fraction, hydroprocessing the wax fraction, and distilling the hydroprocessed products to isolate product streams in the naphtha and middle distillates range. The wax fraction may be recycled to extinction. The hydroprocessing may comprise hydrocracking, hydroisomerization, and/or hydrotreating, as depending upon the characteristics desired in the final product.

Fischer-Tropsch processes may be carried out in fixed bed reactors in which the reactant gas mixture is passed over a bed of solid catalyst, which is packed into tubes, at prescribed ranges of temperature and pressure. A typical arrangement comprises a plurality of small-diameter tubes of several meters in length within a reactor shell. Due to the high exothermicity of the Fischer-Tropsch synthesis, all Fischer-Tropsch reactor designs require adequate provisions for temperature control to prevent reaction runaway. In fixed bed reactors, excess heat is carried away by forming steam from water, which is constantly being added to the reactor shell surrounding the tubes. Fixed bed reactors have several disadvantages that include a complexity that results in high capital costs, a change in catalyst activity over time that compels a catalyst replacement that necessarily takes the reactor off line, heterogeneous axial and radial temperature profiles, and heat removal difficulties.

Fluidized bed reactors differ in ways that give them advantages over fixed bed reactors. Fluidized bed reactors for low-temperature Fischer-Tropsch reactors are also called slurry bubble column, slurry bed, slurry phase, or multiphase reactors, and consist of a shell fitted with cooling coils that run through and project into a slurry comprising catalyst particles and a liquid phase containing hydrocarbons. The reactant gas mixture is introduced to the slurry at the bottom of the reactor shell via a distribution system that creates small gas bubbles that rise up through the slurry. In order for a reaction to occur, the reactant gases must diffuse from the gas bubbles through the liquid phase in the slurry to reaction sites on the suspended catalyst particles, under conditions of sufficient temperature and pressure for the various hydrocarbons and water to be produced. The heavier hydrocarbons are liquid under typical reactor operating temperatures and pressures and are incorporated into the slurry, whereas the lighter gaseous products and water vapor diffuse through and out of the slurry and are eventually collected along with unreacted reactant gas in the reactor head space above the slurry bed level. The collected gas is removed via a gas outlet, and a separation can permit the recovery of unconverted reactants, which can then be recycled either partially or totally to the same synthesis reactor from which the unconverted synthesis gas is recovered and/or to any other synthesis reactor in a multi-staged reactor system.

Fluidized bed reactors have marked advantages over fixed bed reactors for Fischer-Tropsch synthesis. Dispersion of the gas in small bubbles and proper selection of gas flow rates effective in maintaining the catalyst in suspension give rise to a thoroughllymixed slurry, which typically allows the temperature to be consistent throughout. As a result, fluidized bed reactors can tolerate higher average operating temperatures than fixed bed reactors without such problems as excessive catalyst deactivation and coke formation. Because of the nature of the fluidized bed process, catalyst additions and catalyst regenerations can be done online while the reactor is operating in continuous mode without causing a shut-down in operation. Thus, the need to cease operations to regenerate used or spent catalyst or to disassemble the reactor to replace old catalyst, which must be done for fixed bed reactors, is precluded.

Fischer-Tropsch catalysts typically comprise at least one primary catalytic metal from Groups 8, 9, or 10 of the Periodic Table of the Elements (according to the New Notation IUPAC Form as illustrated in, for example, the CRC Handbook of Chemistry and Physics, 82^{nd} Edition 2001-2002; said reference being the standard herein and throughout). Iron, cobalt, ruthenium, and/or nickel are among the commonly preferred metals. Additionally, the catalyst may comprise at least one promoter typically chosen from the group comprising ruthenium, rhenium, platinum, palladium, silver, lithium, sodium, copper, boron, manganese, and potassium. Fischer-Tropsch catalysts may be supported or unsupported. Typical catalyst supports used in Fischer-Tropsch catalysts include any one or any combination of stabilized, modified or unmodified metal oxides, such as silica, ceria, alumina, titania, thoria, boria, zirconia, or combinations thereof.

Catalytic metals used in the Fischer-Tropsch synthesis are usually active for hydrocarbon synthesis in the zero-valent metallic state. Reduced catalytic metals are highly susceptible to oxidation by any number of oxidizing agents, particularly by molecular oxygen in air. Oxidation of the reduced catalytic metals to an oxidized state decreases the catalyst activity, oftentimes to the point that the catalyst has to be regenerated or replaced. Techniques of catalyst preparation typically include steps to reduce the catalytically active metal to a zero-valent state at some time prior to its use as a hydrocarbon synthesis catalyst. Therefore, protection for the catalyst in the reduced state from any oxidation, such as by oxygen from air, is therefore critical. Consequently, techniques of catalyst reduction (also called catalyst activation) and of catalyst loading into a reactor have been developed that reflect this sensitivity.

A typical technique for reducing catalysts used in fixed-bed processes has been in situ reduction in a Fischer-Tropsch reactor using a stream of hydrogen gas followed by introduction of the reactant gas mixture to begin hydrocarbon synthesis. Alternatively, the catalystmay be reduced outside the Fischer-Tropsch synthesis reactor, or even offsite, at a location distant from the hydrocarbon synthesis process. Off-site reduction has been used for catalysts employed in fluidized bed processes.

Since the reduced catalyst is sensitive to oxidation, especially by oxygen present in air and/or by water, it must be protected from such oxidative degradation, and special techniques of catalyst preparation reflect this sensitivity. One way to retain the catalytic metal in a zero-valent state is to embed the catalyst particles in a coating material, for example wax and/or oil, thereby adding a barrier to permeation of an oxidizing agent to the zero-valent metal sites. The coating step is typically done by embedding the reduced catalyst in hydrocarbon wax; most often although not necessarily, a hydrocarbon wax derived from a hydrocarbon synthesis process. Preferably, paraffin waxes from a Fischer-Tropsch process are used. Some in the art form the catalyst/wax mixture into pastilles, pellets or flakes, with the cooled, solid wax protecting the catalyst particles from oxidation.

There are some notable disadvantages associated with this process. The equipment available for the pastillation process is usually not completely airtight and some contact with air during pastillation is unavoidable. Furthermore, the catalyst fabrication facilities may be far away from the hydrocarbon synthesis sites. In addition, packaging, transportation and handling may subject the catalyst pellets to breakage and contact with an oxidizing environment, such as exposure to oxygen contained in air. Moreover, the cost of transporting the catalyst pellets also includes paying for the transport of the coating material (wax) and for the coating step during production at the catalyst fabrication site and then the cost of the transport of heavily coated catalyst to the Fischer-Tropsch plant.

The cost of making hydrogen available at the catalyst manufacturing site is also a disadvantage addressed in the present invention. Once the catalyst/wax pellets reach the hydrocarbon synthesis plant, equipment is needed to handle the pellets and processing the pellets in such a way as to be able to load them into the reactor. The present invention addresses these disadvantages by using the following methods/equipment to prepare and load a hydrocarbon synthesis catalyst in a Fischer-Tropsch reactor.

U.S. Pat. Nos. 5,292,705 and 5,389,690, both to Mitchell, describe a fresh, previously reduced hydrocarbon synthesis catalyst activated by contact with hydrogen at elevated temperatures and pressures and in the presence of liquid hydrocarbons, preferably, sufficient to immerse the catalyst therein.

U.S. Pat. No. 6,512,017 relates to methods of minimizing catalyst degradation during the handling of a catalyst used in a slurry phase reactor. The methods include catalyst handling steps, such as catalyst loading into a slurry phase reactor, slurry phase reactor startup, slurry phase reactor shut-down, and slurry phase reactor unloading when catalyst reloading is envisaged. In the method of loading the slurry phase reactor, a slurry of wax and catalyst is formed in a loading vessel. Clean molten wax is formed in the reactor; syngas is pumped through the clean molten wax in the reactor; and the slurry from the loading vessel is transferred to the reactor.

U.S. Pat. No. 6,515,035 describes a catalyst comprising at least one metal from Groups 8, 9 or 10 of the Periodic Table impregnated on a modified alumina support that is pre-reduced using at least one reducing compound, for example selected from the group formed by hydrogen, carbon monoxide and formic acid, optionally mixed with an inert gas, for example nitrogen, in a reducing compound/(reducing compound+inert gas) mole ratio in the range of 0.001:1 to 1:1. Wherein, the reduction may be carried out in the liquid phase with the catalyst suspended in an inert liquid phase.

The problem of loading a catalyst that is sensitive to spontaneous oxidation of catalytically-active reduced metal, especially by oxygen from air, to a reactor has been addressed previously. One such method features protecting the activated catalyst particles with a layer or coating of some material that can serve as a barrier to permeation of oxygen to the oxidatively sensitive, catalytically-active metal centers on the catalyst. For example, catalyst particles are embedded in a hydrocarbon wax. One inherent disadvantage of the process for embedding the activated catalyst within a wax involves the use of additional equipment to form pastilles, pellets or flakes. The equipment is often not airtight, and the activated catalyst may be exposed to an oxidizing atmosphere (such as air) and at least partially deactivated. In addition, the offsite preparation of protected catalysts necessarily adds to production costs by necessitating the need to transport the catalyst and its waxy protective covering from the off-site catalyst production facility to the reactor facility.

As such, preparing an active Fischer-Tropsch catalyst in which the costs in reduction and transportation are decreased is desirable in leading to overall lower operating costs of the hydrocarbon synthesis process. Consequently, an ongoing need exists to develop more cost effective reduction and loading techniques for making an active Fischer-Tropsch catalyst and for retaining its activity after manufacture. The methods for reducing a metal-based catalyst and for loading it into a Fischer-Tropsch reactor system in accordance with the present invention meets this need, and thus avoids the shortcomings associated with using conventional techniques.

### SUMMARY OF THE INVENTION

The present method features a decrease in transportation costs and catalyst preparation/protection measures in that a catalyst precursor kept in oxide form is brought to the hydrocarbon synthesis facility. Such an oxide form protects the catalyst for transport in an oxidizing environment, such as in air, without any special precautions being taken before and during such transportation. By the present method, a two-fold lessening of transportation costs is realized: the catalyst precursor is transported without a protective coating, which lowers the associated shipping weight thus reducing the transportation cost of the catalyst to the synthesis plant; and the cost of transporting the coating material to the catalyst manufacturing facility is precluded. Increased handling of the catalyst, even in a protected state, increases the chances and degree of potential deactivating exposure to an oxidative atmosphere. Therefore, a catalyst, which comprises a catalytic metal in an oxide form, is safely transported in an oxidizing environment to a synthesis site, without any special precautions being taken before and during transport. The catalyst is received at the hydrocarbon synthesis plant site in its oxide form without any protective coating such as a wax layer. The step of catalyst reduction is then performed at the hydrocarbon synthesis plant site in a reduction vessel with a reducing gas. The reducing gas may comprise one or more of the following gases: hydrogen, nitrogen, carbon dioxide, carbon monoxide, any gaseous hydrocarbon with 5 carbon atoms or less (C₁-C₅ hydrocarbons), natural gas, and any inert gas. The reducing gas preferably comprises hydrogen. The reducing gas may also comprise a small concentration of water or molecular oxygen, preferably not exceeding 10,000 ppm and 1,000 ppm by volume, respectively. The reduction step can be performed in a fluidized bed, an ebulliating bed, or a fixed bed. If a hydrocarbon synthesis catalyst is to be used in a fluidized bed, the reduction step is preferably carried out in a fluidized bed.

Once the reduction is completed, the reduced catalyst is mixed with a hydrocarbon liquid under a non-oxidizing atmosphere. The non-oxidizing atmosphere comprises at least one gas selected from the group consisting of hydrogen, nitrogen, an inert gas, natural gas, any gaseous hydrocarbon comprising 1 to 5 carbon atoms (C₁-C₅ hydrocarbon), and any mixtures thereof. The liquid hydrocarbon can comprise any hydrocarbon with 20 or more carbon atoms, wax, base oil, paraffin oil, or combinations thereof. In some embodiments, the hydrocarbon liquid comprises mostly paraffins. Prior to being mixed with the catalyst, the liquid hydrocarbon is preferably stripped, i.e., contacted with a stripping gas. Contacting the hydrocarbon liquid with the stripping gas should displace the majority of dissolved molecular oxygen (DO₂) out of the hydrocarbon liquid, such that the stripped hydrocarbon liquid is preferably substantially free of dissolved oxygen. The stripping step can take place in a stripping vessel such as a hydrocarbon liquid drum or a melt drum or a pre-operational hydrocarbon synthesis reactor. The stripping gas comprises at least one gas selected from the group consisting of hydrogen, nitrogen, an inert gas, natural gas, and any C₁-C₅ hydrocarbon. An inert gas is defined as a gaseous compound that does not participate (unreactive) in the synthesis reaction by itself with the catalyst and is not deleterious or a poison to the synthesis reaction. The hydrocarbon liquid may be further heated so as to maintain it in a liquid phase. This step is preferably used when the hydrocarbon liquid comprises a wax. The stripping vessel and the mixing vessel therefore may comprise a means of supplying heat. The hydrocarbon liquid could be generated by heating a solid or a partially-solid hydrocarbon matrix at a temperature above the melting point temperature of said matrix.

The reduced catalyst and stripped hydrocarbon liquid are preferably mixed in a mixing vessel to form a catalyst slurry. The mixing vessel can be the stripping vessel in which the hydrocarbon liquid is optionally heated and/or melted and is contacted with a stripping gas; or the reduction vessel in which the catalyst is reduced; or a hydrocarbon synthesis reactor; or a different vessel. When the mixing takes place in the stripping vessel, the method further includes transferring the reduced catalyst under non-oxidizing conditions to the stripping vessel comprising the stripped hydrocarbon liquid. Transferring the reduced catalyst preferably takes place in the presence of at least one gas selected form the group consisting of hydrogen, nitrogen, any (C₁-C₅) gaseous hydrocarbon with 5 carbon atoms or less, natural gas, any inert gas, and mixtures thereof. When the mixing takes place in the reduction vessel, the method further includes transferring at least a portion of the stripped hydrocarbon liquid to the reduction vessel comprising the reduced catalyst. When the mixing takes place in a mixing vessel that differs from the reduction vessel and the stripping vessel, in such an embodiment, at least a portion of the stripped hydrocarbon liquid is transferred to the mixing vessel, and the reduced catalysts are then loaded into the mixing vessel pre-filled with the stripped hydrocarbon liquid.

The catalyst slurry is preferably maintained at a temperature within the typical temperature range of the hydrocarbon synthesis, which is usually from about 160° C. to about 300° C. In some embodiments, the slurry is maintained at a temperature sufficiently high to maintain the hydrocarbon in a liquid phase. In addition, the catalyst particles in the slurry are maintained in a fluidized or suspended state in the hydrocarbon liquid to avoid slumping or compacting of the catalyst particles at the bottom of the mixing vessel. Therefore, the method further includes fluidizing the catalyst slurry so as to distribute the reduced catalyst throughout the hydrocarbon liquid.

The mixing vessel may comprise a temperature-controlling means and an agitating means. The agitating means is effective for suspending the reduced catalyst in the substantially O₂-free hydrocarbon liquid. The agitating means may comprise a system for dispersing a fluidization gas into bubbles throughout the mixing vessel. To maintain the catalyst particles in a fluidized state, the fluidization gas can be fed at the bottom of the mixing vessel, with the formed gas bubbles flowing upwards. The flow rate of the fluidization gas is selected so as to maintain all the catalyst particles dispersed in the slurry. The fluidization gas comprises at least one gas selected from the group consisting of inert gases, nitrogen, hydrogen, natural gas, any gaseous hydrocarbon with 5 or less carbon atoms, inert gas, carbon dioxide, carbon monoxide, synthesis gas, and combinations thereof. The fluidization gas is preferably substantially free of O₂, i.e., when the concentration of O₂ in the fluidization gas is not greater than 1,000 ppm by volume, preferably lower than 100 ppm by volume, more preferably lower than 50 ppm by volume. More preferably, the fluidization gas contains natural gas and/or at least one of the C₁-C₅ hydrocarbons.

The temperature-controlling means of the mixing vessel may comprise internal heating elements such as heating coils or tubes that may circulate a heating medium such as, but not limited to, high pressure steam, so that the catalyst slurry can be maintained at any desired temperature either above, within, or below the Fischer-Tropsch reaction temperature range. The temperature of the slurry in the mixing vessel is selected so as to maintain the hydrocarbon liquid in a liquid form. Once the desired temperature of the slurry is achieved, the slurry may be transferred to an empty reactor or a reactor partially filled with some of the substantially O₂-free hydrocarbon liquid or with a hydrocarbonaceous liquid, preferably by establishing a pressure differential between the mixing vessel and the reactor. The slurry can also be transferred to an on-line reactor that is performing a Fischer-Tropsch synthesis. Alternatively, the mixing step to form the catalyst slurry can be performed in a pre-operational reactor vessel, with transfer of the slurry not necessary.

The method further comprises contacting a reactant gas comprising hydrogen and carbon monoxide under conversion promoting conditions with at least a portion of said catalyst slurry to convert at least a portion of the reactant gas to hydrocarbons. The conversion preferably takes place in at least one hydrocarbon synthesis reactor, which comprises a slurry bubble column. If the mixing step and the conversion steps are performed in different vessels, the portion of the catalyst slurry is transferred preferably under non-oxidizing conditions to the hydrocarbon synthesis reactor.

The catalyst reduction vessel may be one vessel or a plurality of vessels. The stripping vessel where the hydrocarbon liquid is contacted with a stripping gas may also be one vessel or a plurality of vessels. In addition, the vessel where the mixing of the reduced catalyst and substantially O₂-free hydrocarbon liquid takes place can be one vessel or a plurality of vessels. Further, the vessel where the catalyst in the slurry is maintained in the fluidized or suspended state can be one vessel or a plurality of vessels. Moreover, such mixing vessel can be a reactor vessel. The catalyst reduction vessel(s) and the slurry mixing vessel(s) can be sized to supply from about 1% up to about 100% of the catalyst load for one Fischer-Tropsch reactor or up to about 100% of the load for more than one reactor.

In addition, the reduction step, the mixing step, and the conversion step are preferably performed in the proximity of each other. In one embodiment, the reduction step, the mixing step, and the conversion step are performed in the same vessel (i.e., a reactor vessel), so that no transfer is necessary. If a transfer is necessary, for example when the reduction step and the mixing step are done in separate vessel, or when the conversion step and the mixing step are done in separate vessels, the transfer can be done via one or more pipes or conduits, so that the separate vessels are in fluid communication with each other. The transfer can be performed by pressure differential between the separate vessels and/or by a pump.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION OF THE INVENTION

The present invention discloses a method of making a catalyst slurry and loading the same to a hydrocarbon synthesis reactor. The catalyst slurry comprises a mixture of a reduced catalyst and a stripped hydrocarbon liquid comprising wax, paraffin oil, base oil, any hydrocarbon with 20 or more carbon atoms, or mixtures thereof. In some embodiments, the stripped hydrocarbon liquid comprises mostly paraffins. The reduced catalyst retains a high degree of catalytic activity in the catalyst slurry due to its dispersion in the stripped hydrocarbon liquid under non-oxidizing conditions. The stripped hydrocarbon liquid is preferably obtained by contact with a stripping gas to remove most of the dissolved oxygen before being mixed with the reduced catalyst. Moreover, the present method can further preserve the catalytic activity of the reduced catalyst by introducing said slurry to a hydrocarbon synthesis reactor under non-oxidizing conditions. When the hydrocarbon liquid comprises wax, preferably at least a portion of said wax is produced in a hydrocarbon synthesis reactor.

Throughout the specification, the term ‘base oil’ represents a highly-paraffinic oil, which is substantially free of aromatics and polar compounds. A base oil is typically well-suited for the production of engine oils, transmission and gear fluids, hydraulic fluids, and turbine oils. Examples of commercially available base oils can be purchased under the trademarks PURE PERFORMANCE® and ULTRA-S™ Base Oils from ConocoPhillips Company (Houston, Tex.).

In the description below, it shall be understood that the invention may include other elements that are not explicitly recited such as condensers, pumps, separators and recycle loops, the addition of which would be obvious to one of ordinary skill in the art and that fall within the scope of this invention.

The present invention relates to a method of making a catalyst for use in a hydrocarbon synthesis reactor, said method comprising A) providing a catalyst in a reduction vessel, wherein the catalyst comprises particles; B) passing a reducing gas over the catalyst in the reduction vessel under suitable conditions so as to obtain a reduced catalyst; C) providing a hydrocarbon liquid in a stripping vessel; D) contacting the hydrocarbon liquid with a stripping gas in the stripping vessel to generate a stripped hydrocarbon liquid, wherein the stripped hydrocarbon liquid is substantially free of dissolved oxygen; E) mixing the reduced catalyst and at least a portion of the stripped hydrocarbon liquid to provide a slurry; F) fluidizing the slurry so as to distribute the reduced catalyst throughout the stripped hydrocarbon liquid; and G) contacting a reactant gas comprising hydrogen and carbon monoxide under conversion promoting conditions with at least a portion of said slurry to convert at least a portion of the reactant gas to hydrocarbons. The method can be effective for delivering from 1% up to 100% of the catalyst slurry required for at least one hydrocarbon synthesis reactor. Alternatively, the method can be effective for delivering the catalyst slurry in an amount sufficient for more than one hydrocarbon synthesis reactor. Steps (A)-(G) are preferably performed in proximity to each other.

The reducing gas used in reducing step (B) preferably comprises hydrogen but can also comprise one gas selected from the group consisting of nitrogen, carbon dioxide, any C1-C5 light hydrocarbon, natural gas, an inert gas, and mixtures thereof.

The stripping gas used in stripping step (D) preferably comprises a gas selected from the group consisting of nitrogen, hydrogen, carbon dioxide, carbon monoxide, any gaseous hydrocarbon with 5 carbon atoms or less, natural gas, methane, and mixtures thereof. Step (D) is performed for a time period sufficient to achieve a molar fraction of dissolved molecular oxygen less than about 0.1 in the stripped hydrocarbon liquid. Preferably, the stripped liquid hydrocarbons is substantially free of molecular oxygen, i.e., has a molar fraction for dissolved molecular oxygen less than about 0.1.

Fluidization step (F) preferably comprises feeding a fluidization gas comprising at least one gas selected from the group consisting of hydrogen, nitrogen, a gaseous hydrocarbon with 5 carbon atoms or less, natural gas, an inert gas, carbon dioxide, carbon monoxide, and synthesis gas. In some embodiments, step (F) may provide at least one means for mixing in step (E).

If mixing step (E) takes place in the stripping vessel, the method further includes transferring the reduced catalyst under non-oxidizing conditions to the stripping vessel comprising the stripped hydrocarbon liquid. If step (E) takes place in the reduction vessel, the method further includes transferring at least a portion of the stripped hydrocarbon liquid to the reduction vessel comprising the reduced catalyst.

If mixing step (E) takes place in a mixing vessel other than the reduction and stripping vessels, then in such an embodiment, at least a portion of the stripped, hydrocarbon liquid is transferred to the mixing vessel, and the reduced catalyst is then loaded into the mixing vessel pre-filled with the stripped hydrocarbon liquid.

Further, if conversion step (G) takes place in at least one hydrocarbon synthesis reactor, while step (E) and optionally step (F) take place in a different vessel, the method further includes step (H) transferring at least a portion of the slurry made in step (E) to the at least hydrocarbon synthesis reactor. The hydrocarbon synthesis reactor can be empty before the transfer step (H), or partially filled with at least a portion of the stripped hydrocarbon liquid obtained in step (D) before the transfer step (H); or partially filled with a hydrocarbonaceous liquid before the transfer step (H), wherein the hydrocarbonaceous liquid comprises at least one hydrocarbon with 20 or more carbon atoms; a wax; a paraffinic oil; a base oil; or mixtures thereof. In addition, the hydrocarbon synthesis reactor can be in a pre-operational state or in an operating state during the transfer step (H).

Alternatively, the mixing step (E), the fluidization step (F) and the conversion step (G) can take place in at least one hydrocarbon synthesis reactor so that preferably no slurry transfer is necessary.

The method may further comprise heating the hydrocarbon liquid, heating the stripped hydrocarbon liquid, heating the slurry, or combinations thereof.

The stripped hydrocarbon liquid is preferably substantially free of molecular oxygen. The stripped hydrocarbon liquid is formed by a method comprising: optionally, heating a hydrocarbon to obtain a hydrocarbon liquid; and contacting the hydrocarbon liquid with a stripping gas in a manner effective for removing a significant portion of molecular oxygen dissolved in the hydrocarbon liquid. Since the solubility of oxygen is dependent on the hydrocarbon liquid composition as well as temperature and pressure, the effectiveness of the stripping step is best measured by monitoring the dissolved oxygen concentration from the hydrocarbon liquid. The stripping step is effective to achieve a molar fraction of dissolved O₂ in the stripped hydrocarbon liquid below 0.12, preferably below 0.1, more preferably below 0.08. The stripping gas preferably comprises a gas selected from the group consisting of nitrogen, carbon dioxide, carbon monoxide, hydrogen, methane, any gaseous hydrocarbon with 5 carbon atoms or less, natural gas, and mixtures thereof. Contacting the hydrocarbon liquid with the stripping gas is performed for a time period sufficient to achieve a reduced dissolved oxygen concentration in the substantially O₂-free hydrocarbon liquid of not more than 50% of the initial dissolved oxygen concentration in the liquid hydrocarbon.

Alternatively, contacting the hydrocarbon liquid with the stripping gas is performed for a time period of contact between the stripping gas and the hydrocarbon liquid in the stripping vessel sufficient so that the measurable O₂ concentration in the exiting, stripping gas has reached an oxygen concentration value that no longer changes. The maximum concentration value of O₂ in the exiting stripping gas should be below about 500 ppm by weight, preferably below about 100 ppm by weight, and most preferably below about 50 ppm by weight.

The catalyst of step (A) in the present method comprises a catalytic metal wherein a major portion of the catalytic metal exists in at least one oxide form. The catalyst preferably comprises no protective hydrocarbonaceous coating such as a wax layer. As mentioned above, the oxidized catalytic metal sites are catalytically less active than the reduced catalytic metal sites. Often, the oxidized catalytic metal sites are much less active or completely inactive for catalysis. The oxidized catalytic metal is derived from a metal-containing compound, such as a metal salt, a metal oxide, a metal alkoxide, a metal carbonyl, or combination of both. The catalyst manufacturing process may involve conversion of a metal salt to a metal oxide, such as during a calcination step (heating in air). Although it is desirable in the course of the catalyst manufacturing process to convert all of the metal-containing compound to the metal oxide, a small fraction of the metal-containing compound may be present in the oxidized metal catalyst. Although the oxidized metal may be derived from one or more of many metal-containing compounds, preferably the catalyst comprises a catalytic metal in an oxide form. Preferably, the catalyst comprises a catalytic metal from Groups 8, 9, 10 of the Periodic Table or combinations thereof. More preferably, the catalyst comprises at least one metal or metal oxide selected from the group consisting of cobalt, iron, ruthenium, nickel, oxides thereof, and any combination thereof. Most preferably, the catalyst comprises cobalt, wherein at least a portion of the catalytic metal comprising cobalt is in an oxide form.

The present method comprises reducing the catalyst. As explained above, most heterogeneous catalysts are catalytically active only in a reduced state. Preferably, reducing conditions are effective for reducing at least a portion of the oxidized catalytic metal comprised in the catalyst. More preferably, most of the exposed oxidized metal sites are reduced. Preferably, the oxidized catalytic metal is reduced to a catalytically-active oxidation state. More preferably, the catalytically-active oxidation state is the zero-valent metallic state; that is, an oxidation state of zero. In such a preferable embodiment, at least a significant portion of the catalytic metal in oxide form has been reduced to the zero-valent oxidation state. A significant portion of the catalytic metal is reduced to the zero-valent oxidation state when at least 50%, preferably between about 60% and about 80%, of the catalytic metal in oxide form is reduced to the zero-valent oxidation state. In another embodiment, substantially all of the catalytic metal in oxide form is reduced to the zero-valent oxidation state, when between about 80 and 100% of the catalytic metal in oxide form is reduced to the zero-valent oxidation state.

There are many ways to achieve an effective reduction of an oxidized catalytic metal including electrochemical, chemical, and hydrogen gas reduction. A preferred method for reducing an oxidized catalytic metal comprises contacting the catalyst precursor with a reducing gas. The reducing gas may comprise at least one gas selected from the group consisting of hydrogen, nitrogen, carbon dioxide, carbon monoxide, any C₁-C₅ light hydrocarbon, natural gas, any inert gas, and mixtures thereof. The reducing gas may also comprise water with a water content not exceeding about 1 percent by volume (vol %).

Preferably, the reducing gas comprises hydrogen. The reducing gas should have a hydrogen content between about 1 vol % to about 100 vol %, but is preferably rich in hydrogen, with at least about 50 vol % hydrogen. Also according to this specific embodiment, the reducing gas comprising hydrogen may further comprise nitrogen, carbon dioxide, carbon monoxide, any C₁-C₅ light hydrocarbon, natural gas, any inert gas, and mixtures thereof. The reducing gas comprising hydrogen can come from a source within the hydrocarbon synthesis facility or can be imported from elsewhere. Typical sources of hydrogen can be obtained by physical and/or chemical hydrogen enrichment or purification means from a synthesis gas source, from an olefin manufacturing process, from a process for converting hydrocarbons to aromatics; and from a process for converting hydrocarbons to carbon filaments. Physical hydrogen enrichment/purification means from synthesis gas can be performed by membrane separation, pressure swing absorption, or combination thereof, whereas chemical hydrogen enrichment means can comprise a water gas shift reaction, a reforming reaction, or combination, to produce a hydrogen-rich stream. In some embodiments, a gas comprising mostly hydrogen from the hydrocarbon synthesis facility itself and/or from a hydroprocessing facility placed downstream of the hydrocarbon synthesis facility can be recycled to this catalyst reduction step and can make up at least a portion of the reducing gas.

The catalyst comprising an oxidized catalytic metal is reduced under suitable conditions. Suitable conditions comprise, minimally, those capable of reducing at least a portion of the catalytic metal in an oxide form to a catalytically active form. Preferably, the catalytically active form is the zero-valent oxidation state. After the reduction step, the reduced catalyst preferably comprises at least a portion of the catalytic metal in a zero-valent oxidation state. Conditions will vary greatly depending on the particular method of reduction and agents employed. However, when the method comprises contacting the catalyst comprising an oxidized catalytic metal with the reducing gas comprising hydrogen, suitable conditions preferably comprise a temperature between about 200° C. and about 500° C.; preferably a pressure between about 0.1 psig and about 200 psig (about 110-1,480 kPa), more preferably between about 0.1 psig and about 50 psig (about 110-450 kPa); and a reducing gas volumetric flow rate between about 0.1 cubic meters per hour per kilogram of catalyst (m³/hr/kg cat) and about 10 m³/hr/kg cat, preferably between about 0.3 m³/hr/kg cat and about 2 m³/hr/kg cat.

Reduction of the catalyst takes place in a suitable vessel or more than one suitable vessel. Those embodiments employing a gaseous reductant will require a vessel that permits for exposure to the reducing gas at elevated temperatures and mild pressures, wherein “elevated” temperature means greater than 200° C. and “mild” pressure means less than 200 psig (1,480 kPa). Although many vessels may be found suitable in the present invention, preferred vessels are fluidized bed, fixed bed, and rotary kilns. A fluidized bed for reduction is particularly preferred. An alternate embodiment includes reduction taking place in a slurry bed. In other alternative embodiments, the reduction takes place in at least one or a plurality of vessels. The reduction step also comprises producing water. Therefore, the reduction step further comprises generating a reduction effluent comprising water and unused reducing gas. The reduction vessel may further comprise a separation unit to separate water from the reduction effluent, and recover unused reducing gas from the reduction effluent. A gas recycle loop can return at least a portion of the recovered, unused reducing gas to the gas distribution system of the reduction vessel. A preferred separation unit to collect at least a portion of said produced water comprises a cooling device such as a condenser.

Reduction of the catalyst comprising a catalytic metal in oxide form provides a reduced catalyst that is activated for hydrocarbon synthesis. The primary catalytic metal of the reduced catalyst is the reduced form of the catalytic metal described above. The reduced catalyst may further comprise at least one promoter suitable for increasing the selectivity, stability, and/or activity of the reduced catalyst. Suitable promoters are preferably selected from the group consisting of ruthenium, rhenium, platinum, palladium, boron, manganese, silver, lithium, sodium, copper, potassium, and combination thereof. More preferably, when the catalytic metal comprises cobalt, the promoter comprises at least one promoter selected from the group consisting of ruthenium, rhenium, platinum, palladium, silver, boron, and combinations thereof. The reduced catalyst can be supported or unsupported.

Catalyst supports are preferred according to the present invention and provide a platform upon which catalytic components (catalytic metals and promoters) can be deposited. The catalyst support also provides a high porous surface area upon which catalytic components can be distributed. Thus, the catalyst can preferably comprise a support containing an element or one oxide of an element selected from Groups 3, 4, 5, 6, 13, and 14 of the Periodic Table. More preferably, the support comprises an inorganic oxide containing silicon, aluminum, titanium, thorium, boron, zirconium or any combination thereof. Most preferably, the support comprises silica, alumina, titania, or any combination thereof. Alternatively, the support can comprise aluminum in a hydroxide form, such as boehmite or pseudo-boehmite. Also, the support can be treated, stabilized, or modified in order to increase the mechanical/chemical integrity, hydrothermal stability, and/or attrition resistance of the support. Treatment can comprise a high temperature heat treatment, an acid treatment, an alkaline treatment, a steam treatment, a calcination, or combinations thereof. Modification or stabilization is preferred and can be performed by the addition to the support of one or more structural promoters or chemical modifiers. Examples of such structural promoters or chemical modifiers are for example, aluminum, boron, magnesium, silicon, calcium, titanium, chromium, manganese, iron, cobalt, nickel, copper, zinc, gallium, strontium, zirconium, molybdenum, tin, barium, or combinations thereof. The catalyst is preferably comprised of particles. The catalyst preferably comprises particles of weight average particle size between about 30 microns and about 150 microns, more preferably between about 50 microns and about 120 microns.

When the mixing step is not performed in the same vessel as the reduction step, the present method further comprises transferring the reduced catalyst to a mixing vessel. In alternative embodiments, the reduced catalyst is transferred to more than one mixing vessel. The reduced catalyst is sensitive to oxidation, particularly by molecular oxygen, and must be suitably protected from oxygen during transfer. Transferring the reduced catalyst will take place via suitable vessels, tubing and/or piping and under such well-sealed conditions so as to prevent exposure to oxidants, especially molecular oxygen. According to a preferred embodiment, transferring the reduced catalyst takes place under a non-oxidizing atmosphere, which is substantially free of O₂, comprising at least one gas selected from the group consisting of hydrogen, natural gas, any C₁-C₅ light hydrocarbon, nitrogen, and an inert gas. The non-oxidizing atmosphere is substantially free of O₂ when the oxygen content is less than about 1,000 ppm by volume, preferably less than 100 ppm by volume, and more preferably less than 50 ppm by volume.

The invention further comprises mixing the reduced catalyst with a hydrocarbon liquid in one mixing vessel. In alternative embodiments, the reduced catalyst is mixed in moie than one mixing vessel. The mixing vessel should be suitable for mixing the reduced catalyst with the hydrocarbon liquid into a slurry at desired temperatures and pressures. A substantially O₂-free hydrocarbon liquid is preferably used, and, as discussed above, can be obtained by stripping a hydrocarbon liquid with the stripping gas that is essentially free of oxygen. The mixing vessel can further comprise a temperature-controlling means. In particular, when the stripped hydrocarbon liquid comprises wax, the temperature-controlling means is effective for maintaining the wax in a molten state. More particularly, the temperature controlling means comprises one or more heating coils or tubes filled with a heating medium. Steam is a preferred heating medium according to one embodiment of the current invention. In some embodiments, the temperature-controlling means is effective for maintaining the temperature of the slurry within the temperature range used for the hydrocarbon synthesis.

The mixing vessel preferably further comprises an agitating means effective for suspending the reduced catalyst in the hydrocarbon liquid. A preferred agitating means comprises a system for passing a fluidization gas through the slurry in the mixing vessel. Preferred fluidization gases include at least one gas selected from the group consisting of hydrogen, nitrogen, natural gas, any gaseous hydrocarbon with 5 carbon atoms or less such as methane, an inert gas, carbon dioxide, carbon monoxide and synthesis gas. More preferably, the fluidization gas includes hydrogen, nitrogen, natural gas, methane, or combinations thereof. According to one embodiment in which the hydrocarbon liquid comprises wax, the mixing can be performed in a melt drum.

The reduced catalyst is preferably mixed with a substantially O₂-free hydrocarbon liquid to provide a catalyst slurry. The catalyst slurry preferably comprises hydrocarbon synthesis catalyst, and more preferably Fischer-Tropsch catalyst. As discussed above, the preferred method for preparing the substantially O₂-free hydrocarbon liquid comprises optionally heating a hydrocarbon so as to maintain the hydrocarbon in a liquid state in a suitably sealed vessel; and passing the stripping gas through the hydrocarbon liquid in a manner effective for removing substantially all of dissolved molecular oxygen from the hydrocarbon liquid. In this way, it is possible to produce a catalyst slurry that retains a high catalytic activity. In alternative embodiments, the mixing vessel, wherein the reduced catalyst is transferred to and mixed with the substantially O₂-free hydrocarbon liquid, can be the same vessel in which the substantially O₂-free hydrocarbon liquid is prepared. In yet another embodiment, the mixing vessel, wherein the substantially O₂-free hydrocarbon liquid is transferred to and mixed with the reduced catalyst, can be the same vessel in which the reduced catalyst is prepared.

The reduced catalyst and substantially O₂-free hydrocarbon liquid are mixed under suitable conditions of temperature, total pressure and flow rates of the non-oxidizing gas. Suitable conditions preferably comprise a temperature that is within or above the operating range of the hydrocarbon synthesis process. The temperature of the mixing step is preferably between about ambient temperature and about 250° C. More preferably, the temperature is between about 150° C. and about 250° C.

The present method may further comprise transferring the catalyst slurry to at least one fluidizing vessel under conditions sufficient for suspending the reduced catalyst in the substantially O₂-free hydrocarbon liquid prior to introduction of the catalyst slurry to at least one hydrocarbon synthesis reactor. In particular, the fluidizing vessel is effective for preventing slumping or compacting of the reduced catalyst.

The at least one fluidizing vessel comprises a temperature-controlling means and an agitating means, both of which are substantially similar to that described above in connection with the mixing vessel. The fluidization vessel and the mixing vessel can be the same vessel, wherein the mixing is done by the fluidization gas. In addition, the fluidization vessel can be the reactor vessel.

The present method may further comprise transferring the catalyst slurry to at least one reactor vessel, preferably a hydrocarbon synthesis reactor. Although it may be possible to apply the present invention toward a wide range of hydrocarbon synthesis reactors, the preferable application is directed toward fluidized bed hydrocarbon synthesis reactors, particularly toward low-temperature slurry bed hydrocarbon synthesis reactors, wherein the low-temperature synthesis means a temperature range between 190° C. and 280° C. Preferably, the hydrocarbon synthesis reactor product comprises a wax, and at least a portion of said produced wax is used to generate the substantially O₂-free hydrocarbon liquid of the present invention. Preferably, the hydrocarbon synthesis reactor is a Fischer-Tropsch reactor in which a reactant gas mixture comprising carbon monoxide and hydrogen is contacted with at least a portion of the catalyst slurry under suitable conditions of temperature and pressure. Most preferably, the fluidized bed hydrocarbon synthesis reactor is a slurry bubble column reactor. Preferred means for transferring the catalyst slurry comprise a pressure differential between the mixing vessel or fluidizing vessel and the hydrocarbon synthesis reactor.

According to one embodiment, the activated catalyst slurry is transferred to a hydrocarbon synthesis reactor, which is either empty or partially pre-filled with O₂-free hydrocarbon liquid or another hydrocarbonaceous liquid, prior to operation. According to this embodiment, 100% of the catalyst slurry required for the reactor is provided by the invention. According to an alternative embodiment, a fraction of the catalyst slurry required for the reactor is provided. Further according to this embodiment, the catalyst slurry is added to an operational hydrocarbon synthesis reactor. Therefore, according to this embodiment, the method may serve as means for regenerating deactivated catalyst slurry from the reactor and returning activated catalyst slurry back to the reactor in a catalyst slurry recycle loop and/or adding freshly reduced catalyst to a reactor in operation. The present method can be used to generate amounts of fresh catalyst slurry that are sufficient for one reactor or a plurality of reactors.

After the catalyst slurry is loaded into a pre-operational state hydrocarbon synthesis reactor, the catalyst is maintained suspended with a reactor fluidization gas, which comprises a composition the same or different to that of the fluidization gas used in the mixing vessel. This reactor fluidization gas preferably lacks at least one gaseous reactant for the hydrocarbon synthesis reaction, so that no reaction or little reaction takes place while the reactor fluidization gas is flowing. For this reason, the preferred reactor fluidization gas comprises natural gas or at least one C₁-C₅ hydrocarbon. After loading the reactor and after the reactor fluidization gas is introduced, the hydrocarbon synthesis can be started by gradually lowering the reactor fluidization gas flow rate while increasing the reactant gas flow rate, so that the conversion of reactant gas to hydrocarbon products is initiated.

Any of the gases used as reducing gas, mixing vessel fluidization gas, stripping gas, and reactor fluidization gas should have a low concentration of compounds or elements that have a deleterious effect on the catalyst, such as poisons. Any of the gases may need to be pre-treated prior to their respective use to ensure that it contains low concentrations of sulfur or nitrogen compounds such as hydrogen sulfide, hydrogen cyanide, ammonia and carbonyl sulfides. The pre-treatment of these gases can comprise absorption beds, pressure swing adsorption beds, and catalytic beds. In addition, since O₂ and water may cause oxidation of reduced active metal sites, any of the reducing gas, the mixing vessel fluidization gas, the stripping gas, and the reactor fluidization gas could be pre-treated to remove O₂, passed through a condenser, and/or dried preferably at a temperature above 75° C. It is preferable that the O₂ content in any of the reducing gas, the mixing vessel fluidization gas, the stripping gas, and the reactor fluidization gas be below 1,000 ppm by volume; more preferably below 100 ppm by volume; and yet still more preferably below 10 ppm by volume. The water content in any of the reducing gas, the mixing vessel fluidization gas, the stripping gas, and the reactor fluidization gas is preferably below 10,000 ppm by volume; more preferably below 1,000 ppm by volume; and yet still more preferably below 100 ppm by volume.

An alternate method comprises making a catalyst slurry for use in a hydrocarbon synthesis reactor comprising the following steps: a) reducing a catalyst comprising a catalytic metal in an oxide form under suitable conditions to provide a reduced catalyst; b) mixing the reduced catalyst with a substantially O₂-free hydrocarbon liquid under suitable conditions to provide a catalyst slurry; and c) contacting a reactant gas comprising hydrogen and carbon monoxide under conversion promoting conditions in at least one hydrocarbon synthesis reactor comprising at least a portion of said catalyst slurry so as to convert at least a portion of the reactant gas to hydrocarbons, wherein the reduction step, the mixing step and the conversion step are performed in proximity of each other. In one embodiment, all of the steps are performed in the same vessel. In other embodiments, at least two of the steps are performed in separate vessels, which are in fluid communication. When the mixing step and the conversion step are performed in separate vessels, the method further includes d) transferring at least a portion of the catalyst slurry to the at least one hydrocarbon synthesis reactor.

An alternate method comprises a) reducing a catalyst comprising a catalytic metal in an oxide form under suitable conditions in at least one reduction vessel to provide a reduced catalyst; b) transferring the reduced catalyst under non-oxidizing conditions to at least one mixing vessel before mixing; c) mixing the reduced catalyst and a hydrocarbon liquid under suitable conditions in at least one mixing vessel to provide a catalyst slurry; d) optionally, feeding a fluidization gas to the bottom of the mixing vessel under conditions sufficient for suspending the reduced catalyst in the hydrocarbon liquid to avoid slumping or compacting of the catalyst at the bottom of the vessel; and e) optionally, transferring at least a portion of the catalyst slurry to at least one hydrocarbon synthesis reactor. A portion or the totality of said catalyst slurry can be used in one or more hydrocarbon synthesis reactors. The hydrocarbon synthesis reactor can be preoperational (i.e., a start-up stage) or can be in operation mode, wherein a reactant gas is passed through the hydrocarbon synthesis reactor, contacted with the portion or the totality of said catalyst slurry under reaction promoting conditions so as to convert some of the reactant gas to hydrocarbons.

The present invention will include within its scope those variations encompassed by the use of added condensers, pumps, or reactor stages; and a plurality of mixing vessels, fluidizing vessels, reduction vessels and hydrocarbon synthesis reactor vessels.

Fisher Tropsch Conditions:

One preferred embodiment of this method relates to a reduction and loading of a catalyst useful for a hydrocarbon synthesis, also known as the Fischer-Tropsch synthesis. The reactant gas, fed to the Fischer-Tropsch reactor, comprises a mixture of hydrogen (H₂) and carbon monoxide (CO), called synthesis gas. H₂/CO mixtures suitable as a feedstock for conversion to hydrocarbons can be obtained from light hydrocarbons, such as methane or hydrocarbons comprised in natural gas, by means of steam reforming, auto-thermal reforming, dry reforming, advanced gas heated reforming, partial oxidation, catalytic partial oxidation, or other processes known in the art. Alternatively, H₂/CO mixtures can be obtained from biomass and/or from coal by gasification. In addition, the reactant gas can comprise off-gas recycle from the present or another Fischer-Tropsch reactor. Preferably, the hydrogen is provided by free hydrogen, although some Fischer-Tropsch catalysts have sufficient water gas shift activity to convert some water and carbon monoxide to hydrogen and carbon dioxide for use in the Fischer-Tropsch process. It is preferred that the molar ratio of hydrogen to carbon monoxide in the feed be greater than 0.5:1 (e.g., from about 0.67 to about 2.5). Preferably, the reactant gas contains hydrogen and carbon monoxide in a molar ratio of about 1.4:1 to about 2.3:1. The reactant gas can also contain carbon dioxide. The reactant gas stream preferably contains only a low concentration of compounds or elements that have a deleterious effect on the catalyst, such as poisons. For example, the reactant gas may need to be pre-treated to ensure that it contains low concentrations of sulfur or nitrogen compounds such as hydrogen sulfide, hydrogen cyanide, ammonia, and carbonyl sulfides.

The reactant gas is contacted with the activated catalyst slurry in a reaction zone. Mechanical arrangements of conventional design can be employed as the reaction zone including, for example, fluidized bed, slurry bubble column or ebullating bed reactors, among others. Accordingly, the preferred size and physical form of the catalyst particles may vary depending on the reactor in which they are to be used.

The Fischer-Tropsch reactor is typically run in a continuous mode. In this mode, the reactant gas hourly space velocity through the reaction zone typically may range from about 50 to about 10,000 hr⁻¹, preferably from about 300 hr⁻¹ to about 2,000 hr⁻¹. The gas hourly space velocity is defined as the volume of reactants per time per reaction zone volume. The volume of reactant gases is at standard conditions of pressure (101 kPa) and temperature (0° C.). The reaction zone volume is defined by the portion of the reaction vessel volume where the reaction takes place and which is occupied by a gaseous phase comprising reactants, products and/or inert gases; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions with temperatures from about 190° C. to about 260° C.; more preferably from about 205° C. to about 230° C. The reaction zone pressure is typically in the range of about 80 psia (552 kPa) to about 1,000 psia (6,895 kPa), more preferably from 80 psia (552 kPa) to about 800 psia (5,515 kPa), and still more preferably, from about 140 psia (965 kPa) to about 750 psia (5,170 kPa). Most preferably, the reaction zone pressure is from about 250 psia (1,720 kPa) to about 650 psia (4,480 kPa).

Although the present invention and its advantages have been described in detail, it should be understood that various changes, substitutions and alterations may be made herein without departing from the spirit and scope of the invention as defined by the appended claims.

1. A method of making a catalyst for use in a hydrocarbon synthesis reactor comprising:
A) providing a catalyst in a reduction vessel, wherein the catalyst comprises particles;
B) passing a reducing gas over the catalyst in the reduction vessel under suitable conditions so as to obtain a reduced catalyst;
C) providing a hydrocarbon liquid in a stripping vessel;
D) contacting the hydrocarbon liquid with a stripping gas in the stripping vessel to generate a stripped hydrocarbon liquid, wherein the stripped hydrocarbon liquid is substantially free of dissolved oxygen;
E) mixing the reduced catalyst and at least a portion of the stripped hydrocarbon liquid to provide a slurry;
F) fluidizing the slurry so as to distribute the reduced catalyst throughout the stripped hydrocarbon liquid; and
G) contacting a reactant gas comprising hydrogen and carbon monoxide under conversion promoting conditions with at least a portion of said slurry to convert at least a portion of the reactant gas to hydrocarbons.

2. The method according to claim 1 wherein step (E) takes place in the stripping vessel.

3. The method according to claim 2 further including transferring the reduced catalyst under non-oxidizing conditions to the stripping vessel comprising the stripped hydrocarbon liquid.

4. The method according to claim 3 wherein transferring the reduced catalyst takes place in the presence of at least one gas selected form the group consisting of hydrogen, nitrogen, a C₁-C₅ gaseous hydrocarbon with 5 carbon atoms or less, natural gas, any inert gas, and mixtures thereof.

5. The method according to claim 1 wherein step (E) takes place in the reduction vessel.

6. The method according to claim 5 further including transferring at least a portion of the stripped hydrocarbon liquid to the reduction vessel comprising the reduced catalyst.

7. The method according to claim 1 wherein step (G) takes place in at least one hydrocarbon synthesis reactor, and wherein steps (E) and (F) take place in a different vessel.

8. The method according to claim 7 further including
(H) transferring at least a portion of the slurry to the at one least hydrocarbon synthesis reactor.

9. The method according to claim 8 wherein the hydrocarbon synthesis reactor is empty prior to step (H).

10. The method according to claim 8 wherein the hydrocarbon synthesis reactor is partially filled with at least a portion of the stripped hydrocarbon liquid obtained in step (D) before the transfer step (H).

11. The method according to claim 8 wherein the hydrocarbon synthesis reactor is partially filled with a hydrocarbonaceous liquid before the transfer step (H).

12. The method according to claim 11 wherein the hydrocarbonaceous liquid comprises at least one hydrocarbon with 20 or more carbon atoms; a wax; a paraffinic oil; a base oil; or mixtures thereof.

13. The method according to claim 8 wherein the hydrocarbon synthesis reactor is in a pre-operational state.

14. The method according to claim 8 wherein the hydrocarbon synthesis reactor is in an operating state.

15. The method according to claim 1 wherein steps (E), (F) and (G) take place in at least one hydrocarbon synthesis reactor.

16. The method according to claim 1 wherein the catalyst comprises at least one catalytic metal selected from the group consisting of metals from Groups 8, 9, 10 of the Periodic Table.

17. The method according to claim 1 wherein the catalyst comprises at least one catalytic metal in an oxide form.

18. The method according to claim 17 wherein the reduced catalyst comprises at least a portion of the catalytic metal in a zero-valent oxidation state.

19. The method according to claim 1 wherein the catalyst comprises at least one catalytic metal selected from the group consisting of cobalt, iron, ruthenium, nickel, oxides thereof, and combinations thereof.

20. The method according to claim 19 wherein the catalyst further comprises at least one promoter selected from the group consisting of ruthenium, rhenium, platinum, palladium, silver, boron, manganese, lithium, sodium, copper, potassium and combination thereof.

21. The method according to claim 19 wherein the catalytic metal comprises cobalt.

22. The method according to claim 21 wherein the catalyst further comprises at least one promoter selected from the group consisting of ruthenium, rhenium, platinum, palladium, silver, boron, and combinations thereof.

23. The method according to claim 21 wherein the catalyst further comprises a support comprising at least one element selected from the group consisting of silica, alumina, titania, and combinations thereof.

24. The method according to claim 1 wherein the catalyst comprises a support comprising at least one oxide of the elements selected from the group consisting of silicon, aluminum, titanium, thorium, zirconium, boron, and combinations thereof.

25. The method according to claim 1 wherein the catalyst comprises particles of weight average particle size between about 30 microns and about 150 microns.

26. The method according to claim 1 wherein the reducing gas comprises hydrogen.

27. The method according to claim 26 wherein the reducing gas further comprises one gas selected from the group consisting of nitrogen, carbon dioxide, any C₁-C₅ light hydrocarbon, natural gas, an inert gas, and mixtures thereof.

28. The method according to claim 1 wherein the reducing gas has a volumetric flow rate between about 0.1 m³/hr/kg catalyst and about 10 m³/hr/kg catalyst.

29. The method according to claim 1 wherein step (B) is performed at a temperature between about 200° C. and about 500° C.

30. The method according to claim 1 wherein step (B) is performed at a pressure between about 0.1 psig and about 50 psig.

31. The method according to claim 1 wherein the reduction vessel of step (B) is selected from the group consisting of fixed bed, fluidized bed, and rotary kilns.

32. The method according to claim 1 wherein step (B) takes place in a fluidized bed.

33. The method according to claim 1 wherein step (B) takes place in a slurry bed.

34. The method according to claim 1 wherein the reduction vessel comprises a gas recycle loop, and further wherein at least a portion of the gas used in step (B) is recycled through the gas recycle loop to the reduction vessel.

35. The method according to claim 34 wherein step (B) further comprises producing water, and further wherein the method includes removing at least a portion of said produced water from the reducing gas exiting the reduction vessel.

36. The method according to claim 1 wherein the hydrocarbon liquid comprises wax, paraffin oil, base oil, a hydrocarbon with 20 or more carbon atoms, or mixtures thereof.

37. The method according to claim 1 wherein the hydrocarbon liquid comprises wax, and wherein at least a portion of said wax is produced in a hydrocarbon synthesis reactor.

38. The method according to claim 1 wherein the hydrocarbon liquid comprises wax, and further wherein step (D) includes a temperature-controlling means effective for maintaining the wax in a molten state.

39. The method according to claim 1 wherein the hydrocarbon liquid contains mostly paraffins.

40. The method according to claim 1 wherein the stripped hydrocarbon liquid has a molar fraction for O₂ less than 0.12.

41. The method according to claim 1 further comprising heating the hydrocarbon liquid, heating the stripped hydrocarbon liquid, heating the slurry, or combinations thereof.

42. The method according to claim 1 wherein the stripping gas comprises a gas selected from the group consisting of nitrogen, hydrogen, carbon dioxide, carbon monoxide, any gaseous hydrocarbon with 5 carbon atoms or less, natural gas, methane, and mixtures thereof.

43. The method according to claim 1 wherein step (D) is performed for a time period sufficient to achieve a dissolved O₂ content in the stripped hydrocarbon liquid not more than half of the dissolved O₂ content of the liquid hydrocarbon.

44. The method according to claim 1, wherein step (G) is performed in at least one hydrocarbon synthesis reactor comprising a slurry bubble column reactor.

45. The method according to claim 1 wherein step (G) is performed in at least one hydrocarbon synthesis comprising a Fischer-Tropsch synthesis.

46. The method according to claim 1 wherein step (E) comprises feeding a fluidization gas comprising a gas selected from the group consisting of hydrogen, nitrogen, a gaseous hydrocarbon with 5 carbon atoms or less, natural gas, an inert gas, carbon dioxide, carbon monoxide, synthesis gas, and combinations thereof.

47. The method according to claim 1 wherein step (F) provides at least one means for mixing in step (E).

48. The method according to claim 1 wherein the method is effective for delivering from 1% to 100% of the catalyst slurry required for at least one hydrocarbon synthesis reactor.

49. The method according to claim 1 wherein step (E) is performed in more than one vessel.

50. The method according to claim 1 wherein the method is effective for delivering the catalyst slurry in an amount sufficient for more than one hydrocarbon synthesis reactor.

51. The method according to claim 1 wherein steps (A)-(G) are performed in proximity to each other.

52. A method for making a catalyst slurry for use in a hydrocarbon synthesis reactor, comprising:
a) reducing a catalyst comprising a catalytic metal in an oxide form under suitable conditions to provide a reduced catalyst;
b) mixing the reduced catalyst with a substantially O₂-free hydrocarbon liquid under suitable conditions to provide a catalyst slurry; and
c) contacting a reactant gas comprising hydrogen and carbon monoxide under conversion promoting conditions in at least one hydrocarbon synthesis reactor comprising at least a portion of said catalyst slurry so as to convert at least a portion of the reactant gas to hydrocarbons,
wherein steps a), b), and c) are performed in proximity of each other.
